(19) United States
(12) Patent Application Publication
(10) Pub. No.: US 2006/0211937 A1
(43) Pub. Date: Sep. 21, 2006

(54) Garment to facilitate use of a portable monitor device

(76) Inventor: Robert Eldridge, Owosso, MI (US)

Correspondence Address: DICKINSON WRIGHT PLLC, 1901 L. STREET NW, SUITE 800, WASHINGTON, DC, 20036, US

(21) Appl. No.: 11/081,580

(22) Filed: Mar. 17, 2005

(51) Int. Cl. A61B 5/04

(52) U.S. Cl. 600/388

(57) ABSTRACT

A garment configured to hold a portable medical device, and more particularly to a modified upper garment to hold, secure and conceal a heart monitor while allowing easy and unobtrusive access to cardiac lead points on a patient. The garment has an exterior pocket for a monitor. It further has a plurality of openings to allow attachment of monitor leads on a patient without the need to remove the garment. The openings may also have closure means. The garment provides modesty, comfort, durability and an attractive appearance. The garment may be configured for use in all cardiac rehabilitation situations including exercise and stress testing. The entire garment is made of x-ray transparent materials.

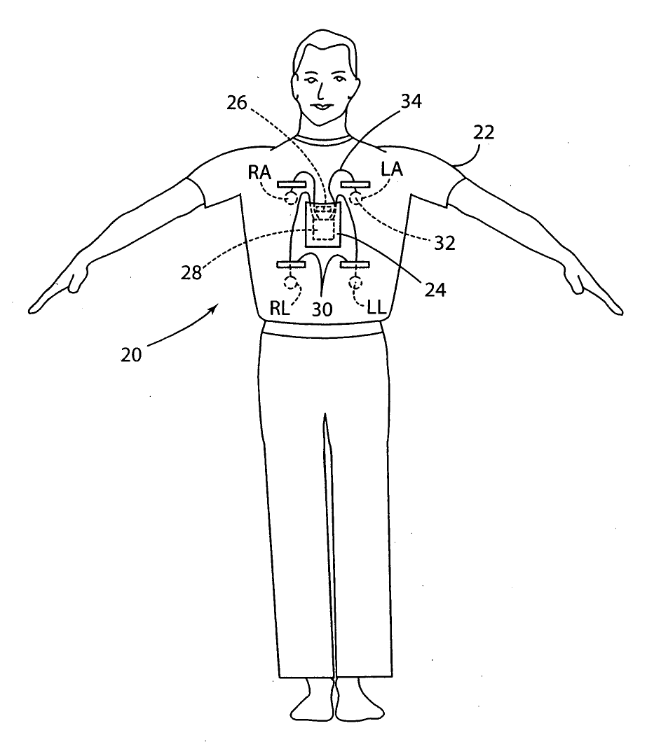

### FIELD OF THE INVENTION

The present invention generally relates to garments configured to hold a portable medical device, and more particularly to a modified upper garment to hold and conceal a heart monitor while allowing easy and unobtrusive access to cardiac lead points on a patient.

### BACKGROUND OF THE INVENTION

Individuals under cardiac treatment or evaluation may require frequent visits to a medical facility to monitor various cardiac and pulmonary functions. During these visits, a patient is often required to be connected to any of a number of cardiac or pulmonary monitors such as an electrocardiograph (ECG), Holter Monitor, pendant, credit card heart monitor, flexible sensor pads, and/or chest strap. Wearing these monitors has undesirable and impractical implications. For example, a patient is often afforded the discomfort and embarrassment of removing their shirt or upper garment to allow attachment of cardiac sensors (electrodes) that lead to a cardiac monitor. Once applied, the patient must either remain seated or hold the heart/cardiac monitor while ambulatory.

There have been attempts in the prior art to reduce or eliminate these problems associated with heart monitor use. Some provide complicated garments that integrate cardiac electrode leads into the fabric of the garment (see generally, U.S. App. 2003/0212319 to Magill; U.S. Pat. No. 6,381,482 to Jayaraman et al.; and U.S. Pat. No. 4,583,547 to Granek et al.). Nevertheless, they do not address the variability of medical devices used and would be difficult to clean or use for any other purpose. Further, these types of garments would appear expensive to manufacture and fragile while in use.

Still, there are attempts in the art to make a garment for use during heart monitoring more comfortable, aesthetically pleasing, and easy to use. U.S. Pat. No. 5,611,085 to Rasmussen discloses a vest configured to hold a cardiac monitor in place on a patient, but sill requires a user to remove his or her shirt to attach the electrode leads and does little to cover the patient making an additional garment placed on top of the vest necessary. Additionally, U.S. Pat. No. 4,698,848 to Buckley discloses a woman's blouse designed to hold a cardiac monitor in an internal pocket. The blouse must also be opened to expose the patient's torso to attach the electrode leads. Unfortunately, Buckley results in a fairly uncomfortable experience for the patient since the monitoring unit and the electrode leads are all between the patient's skin and the garment. Further, Buckley does not address the similar problems associated with men in the same situation.

Thus, there is a desire and a need in the art to provide a universal and unobtrusive garment to hold a portable monitoring unit, such as a heart monitor. The garment should be discreet, attractive, inexpensive and comfortable while allowing easy access to electrode lead points.

### SUMMARY OF THE INVENTION

Accordingly, it is an important aspect of the invention to provide a garment configured to hold a portable medical device, and more particularly a modified upper garment to hold and conceal a heart monitor while allowing easy and unobtrusive access to cardiac electrode lead points on a patient.

In general, this is achieved through the use of a garment such as a normal t-shirt that includes an exterior pocket with a hook and loop fastener such as those sold under the trade name VELCRO or snap closure in the middle of the chest. This pouch holds the heart monitor unit during use. The shirt also has openings positioned at approximate sites where the heart monitor leads are placed on the patient's chest. These openings may also be closable using hook and loop fasteners. The garment may be made from a standard cotton t-shirt or any various other dimensionally stable materials may be used instead and preference should be given to those materials that are transparent to X-rays should the patient require such procedure on their visit to the doctor.

One embodiment of the invention provides a garment to facilitate use of a portable monitor device, in which the garment is configured to cover a chest of a patient and made of an x-ray penetrable material, has at least one exterior pocket configured to retain a monitor means having a closure means made of an x-ray penetrable material, and at least one opening on said garment configured to allow placement of a medical device on the patient, whereby the patient does not need to remove the garment.

Additional embodiments include at least one opening further having a closure means made of an x-ray penetrable material. The closure means may comprise at least one hook and loop closure. The garment may have short sleeves. The at least one opening may also have four openings in an arrangement to allow access to heart monitor electrodes.

Additional aspects and advantages of the invention will become apparent from the following detailed description, the drawings, and the appended claims.

### BRIEF DESCRIPTION OF THE FIGURE

The foregoing features, as well as other features, will become apparent with reference to the description and figures below, in which like numerals represent like elements, and in which the Figure is a frontal view of the present invention on a patient.

### DETAILED DESCRIPTION OF THE INVENTION

The present invention generally relates to a garment configured to hold a portable medical device, and more particularly to a modified upper garment to hold, secure, and conceal a heart monitor while allowing easy and unobtrusive access to cardiac lead points on a patient. One embodiment of the present invention provides a garment to facilitate use of a portable monitor device that is easy to use and convenient to wear. This is achieved through the use of a garment such as a normal t-shirt that includes an exterior pocket with a hook and loop fastener such as those sold under the trade name VELCRO or snap closure in the middle of the chest. This pouch holds the heart monitor unit during use. The shirt also has openings positioned at approximate sites where the heart monitor leads are placed on the patient's chest. These openings may also be closable using hook and loop fasteners. The garment may be made from a standard cotton t-shirt or any various other dimensionally stable materials may be used instead and preference should be given to those materials that are transparent to X-rays should the patient require such procedure on their visit to the doctor.

Referring now to the Figure, a garment 20 configured to cover a chest of a person for a cardiac patient is illustrated. The garment 20 is configured to cover the chest of a person and has an exterior pocket 24 and short sleeves 22. It is noted that any type of sleeve suitable for patient comfort and desire is possible within the scope of the present invention. Nevertheless, short sleeves 22 allow for a blood-pressure cuff, such as commonly used during stress testing, to be placed on a patient's arm without difficulty. Further, the loose-fitting short sleeves provide maximum comfort. Further, it is noted that depending on the type of monitoring application, additional exterior or even interior pockets may be applied and fall within the scope of the invention.

Exterior pocket 24 is configured to be large enough to hold a medical device 28 such as a heart or cardiac monitor. Exterior pocket 24 also has a closure means 26 to secure medical device 28. Garment 20 is also configured to have additional openings 30 to allow placement of electrode lead wires 34 through garment 20 for attachment of electrodes 32 to a patient's skin without the need to remove the garment. Once electrodes 32 are placed on the patient, openings 30 may also be closed using a closure means. As shown, a common cardiac electrode configuration includes four electrodes 32 placed on the upper and lower left and upper and lower right of a patient's chest.

The closure means 26 for exterior pocket 24 and openings 30 may be formed of an x-ray penetrable material, such as a hook and loop fastener sold under the trade name VELCRO. The remainder of the garment materials is similarly x-ray transparent. Exterior pocket 24 is located on the outside portion of the front of garment 20. Exterior pocket 24 is of proper size and shape to securely accommodate a cardiac monitor 28. Exterior pocket 24 is located so as to allow electrode leads 34 to be properly connected to conventional electrodes 32.

In normal use, the electrodes 32 are first fastened to the patient's chest while garment 20 is on the patient. Electrode leads 34 are connected to the electrodes 32 and monitor 28 placed in pocket 24. Minimal privacy is needed since garment 20 does not need to be removed.

Typically, during stress testing, a monitor is not worn, but the patient's blood pressure is monitored. The short sleeves of the garment of the invention allow convenient placement of the blood pressure cuff.

While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

The invention claimed is:

1. A garment to facilitate use of a portable monitor device, comprising:
a garment configured to cover a chest of a patient and made of an x-ray penetrable material;
at least one exterior pocket configured to retain a monitor means having a closure means made of an x-ray penetrable material; and
at least one opening on said garment configured to allow placement of a medical device on the patient, whereby the patient does not need to remove the garment.

2. The garment of claim 1, wherein said at least one opening further comprises a closure means made of an x-ray penetrable material.

3. The garment of claim 1, wherein said closure means comprises at least one hook and loop closure.

4. The garment of claim 2, wherein said closure means comprises at least one hook and loop closure.

5. The garment of claim 1, further comprising short sleeves.

6. The garment of claim 1, wherein said at least one opening is comprised of four openings in an arrangement to allow access to heart monitor electrodes.

7. A garment for cardiac patients comprising:
a garment configured to cover a chest of a patient and made of an x-ray penetrable material;
at least one exterior pocket configured to retain a monitor means having a closure means made of an x-ray penetrable material; and
at least one opening on said garment configured to allow placement of a medical device on the patient, whereby the patient does not need to remove the garment.

8. The garment of claim 6, wherein said at least one opening has a closure means made of an x-ray penetrable material.

9. The garment of claim 6, wherein said closure means comprises at least one hook and loop closure.

10. The garment of claim 7, wherein said closure means comprises at least one hook and loop closure.

11. The garment of claim 6, further comprising short sleeves.

12. The garment of claim 6, wherein said at least one opening is comprised of four openings in an arrangement to allow access to heart monitor electrodes.
